(19) United States
(12) Patent Application Publication
(10) Pub. No.: US 2016/0175766 A1
(43) Pub. Date: Jun. 23, 2016

(54) METHOD OF PURIFYING AN IONIC LIQUID AND METHOD OF DEHUMIDIFYING AIR

(71) Applicant: Evonik Degussa GmbH, Essen (DE)

(72) Inventors: Olivier Zehnacker, Dortmund (DE); Benjamin Willy, Dusseldorf (DE); Xinming Wang, Kanagawa-ken (JP); Rolf Schneider, Grundau-Rothenbergen (DE); Marc-Christoph Schneider, Offenbach (DE)

(73) Assignee: Evonik Degussa GmbH, Essen (DE)

(21) Appl. No.: 14/973,084

(22) Filed: Dec. 17, 2015

(30) Foreign Application Priority Data
Dec. 18, 2014 (DE) 102014226441.3

(51) Int. Cl. B01D 53/26; C07C 53/10; C07F 9/06; C07D 233/58; C07C 309/04

(52) U.S. Cl. CPC B01D 53/263; C07D 233/58; C07C 309/04; C07F 9/06; C07C 53/10; B01D 2252/30; B01D 2252/20473

(57) ABSTRACT

Ionic liquids of the structure Q⁺A⁻, in which Q⁺ is a 1,3-dialkylimidazolium ion, in which the alkyl groups, independently of one another, are linear C₁-C₄-alkyl groups and A⁻ is the anion of an acid HA with a pKₐ of less than 3, can be purified by desorption of volatile compounds at a temperature of 100 to 200° C. and a pressure of at most 100 mbar over a period of at least 0.1 h and be used for dehumidifying air.

### CROSS REFERENCE TO RELATED APPLICATIONS

The present application claims priority to German Application, DE 102014226441.3, filed on Dec. 18, 2014, the contents of which is incorporated herein by reference in its entirety.

### FIELD OF THE INVENTION

The invention relates to a method of purifying an ionic liquid and a method of dehumidifying air using the purified ionic liquid.

### BACKGROUND OF THE INVENTION

In air conditioning systems for the aeration and conditioning of buildings or vehicles, air generally not only has to be cooled, but also dehumidified. This is because the air often has such a high humidity that, upon cooling, its temperature falls below the dew point. Hence in conventional air conditioning systems dehumidification of the air accounts for a large part of electricity consumption.

One option for reducing the electricity consumption of air conditioning systems for buildings is the dehumidification of air by adsorption or absorption of water using a drying medium which is then regenerated from the water laden product by heating to a temperature at which the water is desorbed. Compared to adsorption on a solid absorbent, the advantages of absorption in a liquid absorption medium are that drying can be performed with less complex equipment, that less drying medium is required, and that regeneration of the water-laden drying medium using solar heat is easier to carry out.

Aqueous solutions of lithium bromide, lithium chloride or calcium chloride, hitherto employed as liquid absorption media in commercial air conditioning systems, have the disadvantage that they are corrosive towards the metallic materials typically employed in air conditioning systems and expensive alternative materials are needed to avoid this corrosion. In addition, these solutions can cause problems due to salt crystallizing out of the absorption medium.

Y. Luo et al., Appl. Thermal Eng. 31 (2011) 2772-2777 proposes the ionic liquid 1-ethyl-3-methylimidazolium tetrafluoroborate in place of aqueous solutions of lithium bromide for drying of air.

Y. Luo et al., Solar Energy 86 (2012) 2718-2724 proposes the ionic liquid 1,3-dimethyimidazolium acetate as an alternative to 1-ethyl-3-methylimidazolium tetrafluoroborate for drying of air.

US 2011/0247494 Al proposes, in paragraph [0145], the use of trimethylammonium acetate or 1-ethyl-3-methylimidazolium acetate as liquid drying agent instead of aqueous lithium chloride solution. Example 3 compares water uptake from humid air for a series of further ionic liquids.

CN 102335545 A describes aqueous solutions of ionic liquids as absorption media for air dehumidification. The ionic liquids can contain the anions [BF₄]⁻, [CF₃SO₃]⁻, [CH₃COO]⁻, [CF₃COO]⁻, [C₃F₇COO]⁻, [(CF₃SO₂)₂N]⁻, [(CH₃)₂PO₄]⁻, [C₄F₉SO_{3]}⁻, [(C₂F₅SO₂)N]⁻ and [(CF₃SO₂)₃C]⁻.

### DESCRIPTION OF THE INVENTION

It has now been found that commercially available ionic liquids generally comprise impurities which lead to substances that are odour-intensive or are injurious to health and that these may enter dehumidified air in dehumidification procedures using the ionic liquids. Moreover, it has been found that, during the desorption of water from ionic liquids which contain a basic anion, e.g., a carbon/late ion, odour-intensive decomposition products are formed. If the ionic liquids are subsequently used in a dehumidification procedure, the decomposition products may enter into the dehumidified air. It has also been found that, by desorption of volatile compounds at 100 to 200° C. and a pressure of less than 100 mbar, a purified ionic liquid can be produced from an ionic liquid with a non-basic or weakly basic anion. When used to dehumidify air, this purified ionic liquid exhibits improved characteristics with respect to the release of odour-intensive or unhealthy substances.

The invention therefore provides a method of purifying an ionic liquid, in which volatile compounds are desorbed from an ionic liquid of the structure Q⁺A⁻, wherein Q⁺ is a 1,3-dialkylimidazolium ion, in which the alkyl groups, independently of one another, are linear C₁-C₄-alkyl groups and A⁻ is the anion of an acid, HA, with a pKₐ of less than 3. Desorption proceeds at a temperature of 100 to 200° C. and a pressure of, at most, 100 mbar over a period of at least 0.1 h.

The invention is also directed to a method of dehumidification, in which air is brought into contact with an absorption medium comprising an ionic liquid purified using the methods described herein. The ionic liquid has the structure Q⁺A⁻, where Q⁺ is a 1,3-dialkylimidazolium ion, in which the alkyl groups, independently of one another, are linear C₁-C₄-alkyl groups, A⁻ is the anion of an acid HA with a pKₐ of less than 3 and is not a halide ion, and where a mixture of 95% by weight of ionic liquid Q⁺A⁻ and 5% by weight of water at a temperature of 80° C. has a vapour pressure of less than 100 mbar.

The methods according to the invention are carried out with an ionic liquid of the structure Q⁺A⁻. Q⁺ is a 1,3-dialkylimidazolium ion, in which the alkyl groups, independently of one another, are linear C₁-C₄-alkyl groups. Suitable 1,3-dialkylimidazolium ions are 1,3-dimethylimidazolium, 1-ethyl-3-methylimidazolium, 1-propyl-3-methylimidazolium, 1-butyl-3-methylimidazolium, 1,3-diethylimidazolium, 1-ethyl-3-propylimidazolium, 1-butyl-3-ethylimidazolium, 1,3-dipropylimidazolium, 1-butyl-3-propylimidazolium and 1,3-dibutylimidazolium. Preferably, Q⁺ is a 1,3-dimethylimidazolium cation or a 1-ethyl-3-methylimidazolium cation and particularly preferably a 1,3-dimethylimidazolium cation. 1,3-Dialkylimidazolium ions with linear alkyl groups have the advantage over 1,3-dialkylimidazolium ions with branched alkyl groups that a dealkylation during the methods according to the invention is avoided. 1,3-Dialkylimidazolium ions with C₁-C₄-alkyl groups have the advantage over 1,3-dialkylimidazolium ions with longer alkyl groups that they achieve a higher water absorption capacity in an absorption medium during the dehumidification of air.

The ionic liquid of the structure Q⁺A⁻ comprises, as anion A⁻, the anion of an acid HA with a pKₐ of less than 3. The pKₐ refers here to an aqueous solution of the acid HA at 25° C. The anion A⁻ can be the anion of an organic acid or of an inorganic acid. Preferably, the anion A⁻ is hydrogensulphate, methanesulphonate, methylsulphate, ethylsulphate, dihydrogenphosphate, dimethylphosphate, diethylphosphate or nitrate. Particularly preferably, diethylphosphate is the anion A⁻. By using an anion A⁻ of an acid HA with a pKₐ of less than 3 it is possible to reduce the formation of odour-intensive impurities during the purification according to the invention and during subsequent use of the ionic liquid for dehumidifying air. If ionic liquids have an anion of a weaker acid, in particular a carboxylate ion, odour-intensive impurities can form to a greater extent during the purification according to the invention and upon using the ionic liquid for dehumidifying air.

In the method of purifying an ionic liquid described herein, volatile compounds are desorbed from the ionic liquid at a temperature of 100 to 200° C. and a pressure of at most 100 mbar over a period of at least 0.1 h. The desorption of volatile compounds is preferably carried out at a temperature of 120 to 180° C. and particularly preferably from 140 to 160° C. The pressure is preferably 0.01 to 20 mbar and particularly preferably 0.01 to 10 mbar. The desorption of volatile compounds is preferably carried out for a period of from 0.5 to 100 h, and particularly preferably 1 to 10 h. The temperature, pressure and duration of the desorption are chosen according to the amount and type of volatile compounds in the ionic liquid used, with a higher temperature, a lower pressure and/or a longer duration being chosen for larger amounts and for less volatile compounds. Suitable process conditions for the desorption of volatile compounds can be determined by means of a sensory test of the purified ionic liquid as to odour, and also by means of Headspace-GC of the purified ionic liquid.

The rate of the desorption of volatile compounds can be increased by enlarging the surface area of the ionic liquid. Preferably, during desorption, the ionic liquid is passed over a bed of filling materials or over a structured packing. All filling materials and structured packings which are known to the person skilled in the art for distillations and for absorption processes may be used for this purpose. Alternatively, desorption can take place in a falling film apparatus. Suitable falling film apparatuses are falling film evaporators known from the prior art for distillations.

The rate of desorption of volatile compounds can be increased by passing an inert gas at a pressure of at most 100 mbar through the ionic liquid or, in co-current or countercurrent with the ionic liquid, passing it through a bed of filling materials, a structured packing or a falling film apparatus. Suitable inert gases are nitrogen, CO_{2,} water vapour, argon and helium, with nitrogen being preferred.

The ionic liquid purified can be used for dehumidifying air. Preferably, this takes place in a method in which the air is brought into contact with an absorption medium which comprises an ionic liquid with a structure Q⁺A⁻ as defined above, and where a mixture of 95% by weight of ionic liquid Q⁺A⁻ and 5% by weight of water at a temperature of 80° C. has a vapour pressure of less than 100 mbar. The contacting can take place in all apparatuses which are known in the art for gas absorption methods with liquid absorption agents or for the drying of air with aqueous solutions of lithium chloride or lithium bromide. Apparatuses that can be used include those in which the air is passed co-currently or preferably countercurrently to the absorption medium through a bed of filling materials or through a structured packing. Other apparatuses that may be used include those in which the absorption medium trickles or flows away over cooling tubes or cooling fins circulated by air. Preferably, the air is brought into contact with the absorption medium in a falling film apparatus, which makes it possible to avoid the air entraining droplets of the absorption medium.

Ionic liquids which, in a mixture with 5% by weight of water at a temperature of 80° C., have a vapour pressure of less than 100 mbar can be selected by means of routine experiments, preferably from ionic liquids having an anion from the group hydrogensulphate, methanesulphonate, methylsulphate, ethylsulphate, dihydrogenphosphate, dimethylphosphate, diethylphosphate and nitrate. Of particular suitability are the ionic liquids 1,3-dimethylimidazolium hydrogensulphate, 1,3-dimethylimidazolium methanesulphonate, 1,3-dimethylimidazolium ethylsulphate, 1,3-dimethylimidazolium diethylphosphate, 1,3-dimethylimidazolium nitrate, 1-ethyl-3-methylimidazolium hydrogensulphate, 1-ethyl-3-methylimidazolium methanesulphonate, 1-ethyl-3-methylimidazolium ethylsulphate, 1-ethyl-3-methylimidazolium diethylphosphate and 1-ethyl-3-methylimidazolium nitrate.

In the method of the invention, the absorption medium, prior to being brought into contact with air, comprises preferably at least 80% by weight, and particularly preferably more than 85% by weight, ionic liquid of the structure Q⁺A⁻. Preferably, the total content of ionic liquids of the structure Q⁺A⁻ and of water in the absorption medium is more than 90% by weight, and particularly preferably more than 98% by weight.

Absorption medium laden with water during the dehumidification of air can be regenerated again by evaporation of water and be reused for the dehumidification of air. For this purpose, the water-laden absorption medium is preferably heated, preferably to a temperature of 70 to 120° C. and the evaporated water is condensed or led away with a stream of air. The condensation of water can be effected by cooling with water or with air. Preferably, the evaporation of water is carried out in a falling film evaporator and the evaporated water is removed with a stream of air, which is particularly preferably a stream of waste air from an air conditioned building or vehicle.

Using the method according to the invention, air can be dehumidified with small amounts of absorption medium to an extent required for the operation of an air conditioning plant. This can be done without crystallization of absorption agent from the absorption medium and with a reduced release of substances that are odour-intensive or injurious to health into dehumidified air.

### EXAMPLES

Volatile compounds were desorbed from ionic liquids on a rotary evaporator at a temperature of 140° C. and a pressure of 10 mbar over a period of 24 h. The ionic liquids were analysed before and after treatment sensorily as to odour and by means of headspace-GC as to volatile impurities. For the headspace-GC analysis, the ionic liquid was heated to 70° C. in a headspace sample container for 20 min before the air above the ionic liquid was analysed by gas chromatography. The sensory assessment of the odour and the substances detected in the air by means of headspace-GC are listed in Tables 2 and 3. With the exception of the ionic liquid from Example 4, which was acquired from lolitec, the ionic liquids were prepared by condensation of glyoxal, formaldehyde, methylamine and the acid listed in Table 1 in the molar ratio 1:1:2:1 by the method described in WO 2009/074535. In Example 6, the majority of the ionic liquid was broken down to give 1-methylimidazole and methyl acetate during the experiment.

TABLE 1
Ionic liquids investigated

| | | Synthesis and/ |
|---|---|---|
| Example | Ionic liquid | or manufacturer |
| 1 | 1,3-Dimethylimidazolium | Condensation with H₂SO₄ |
| | hydrogensulphate | |
| 2 | 1,3-Dimethylimidazolium | Condensation with |
| | methanesulphonate | CH₃SO₃H |
| 3 | 1,3-Dimethylimidazolium nitrate | Condensation with HNO₃ |
| 4 | 1-Ethyl-3-methylimidazolium | Iolitec |
| | diethylphosphate | |
| 5 | 1,3-Dimethylimidazolium | Condensation with |
| | diethylphosphate** | (EtO)₂P(O)OH |
| 6* | 1,3-Dimethylimidazolium | Condensation with |
| | acetate | acetic acid |

*not according to the invention
**comprises ethyl hydrogenphosphate

TABLE 2
Unpurified ionic liquid

| | Sensory assessment | Volatile substances in the |
|---|---|---|
| Example | of the odour | headspace-GC |
| 1 | Fishy | Not determined |
| 2 | Fishy | Not determined |
| 3 | Fishy | Not determined |
| 4 | Weak | Acetonitrile, dichloromethane |
| 5 | Strong fishy | 1,2-Diaminoethane, |
| | | trimethylamine, ethanol, acetone, |
| | | 2-aminobutane, triethylphosphate |
| 6* | Strong fishy | 1,2-Diaminoethane, methyl |
| | | acetate, acetic acid |

*not according to the invention

TABLE 3
Purified ionic liquid

| | | Volatile substances in the |
|---|---|---|
| Example | Sensory evaluation of the odour | headspace-GC |
| 1 | Weak, sweetish | none |
| 2 | Weak, sweetish | Ethanol |
| 3 | Weak, fishy | none |
| 4 | Weak | Ethanol, triethylphosphate |
| 5 | Weak, sweetish | Ethanol, triethylphosphate |

Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A method of purifying an ionic liquid, comprising desorbing volatile compounds from an ionic liquid of the structure Q⁺A⁻, wherein:
Q⁺ is a 1,3-dialkylimidazolium ion, in which the alkyl groups, independently of one another, are linear C₁-C₄-alkyl groups; and
A⁻ is the anion of an acid, HA, with a pKₐ of less than 3;
and wherein said desorbing is performed at a temperature of 100 to 200° C. and a pressure of at most 100 mbar over a period of at least 0.1 hour.

2. The method of claim 1, wherein A⁻ is selected from hydrogensulphate, methanesulphonate, methylsulphate, ethylsulphate, dihydrogenphosphate, dimethylphosphate, diethylphosphate and nitrate.

3. The method of claim 1, wherein Q⁺ is a 1,3-dimethylimidazolium cation or a 1-ethyl-3-methylimidazolium cation.

4. The method of claim 1, wherein desorption takes place over a period of from 0.5 to 100 h.

5. The method of claim 1, wherein, during the desorption, the ionic liquid is passed over a bed of filling materials or over a structured packing.

6. The method of claim 1, wherein desorption takes place in a falling film apparatus.

7. A process for dehumidifying air comprising purifying an ionic liquid by the method of claim 1.

8. A method for dehumidifying air, comprising:
a) purifying an ionic liquid Q⁺A⁻, by the method of claim 1;
b) bringing air into contact with an absorption medium comprising the purified ionic liquid of the structure Q⁺A⁻, wherein:
Q⁺ is a 1,3-dialkylimidazolium ion, in which the alkyl groups, independently of one another, are linear C₁-C₄-alkyl groups,
A⁻ is the anion of an acid HA with a pKₐ of less than 3 and is not a halide ion,
and wherein a mixture of 95% by weight of ionic liquid Q⁺A⁻ and 5% by weight of water at a temperature of 80° C. has a vapour pressure of less than 100 mbar.

9. The method of claim 8, wherein the ionic liquid is selected from the group consisting of: 1,3-dimethylimidazolium hydrogensulphate; 1,3-dimethylimidazolium methanesulphonate; 1,3-dimethylimidazolium ethylsulphate; 1,3-dimethylimidazolium diethylphosphate; 1,3-dimethyimidazolium nitrate; 1-ethyl-3-methylimidazolium hydrogensulphate; 1-ethyl-3-methylimidazolium methanesulphonate; 1-ethyl-3-methylimidazolium ethylsulphate; 1-ethyl-3-methylimidazolium diethylphosphate; and 1-ethyl-3-methylimidazolium nitrate.

10. The method of claim 8, wherein the absorption medium, prior to being brought into contact with the air, comprises at least 80% by weight of ionic liquid of the structure Q⁺A⁻.
